(19) United States
(12) Patent Application Publication
(10) Pub. No.: US 2017/0265846 A1
(43) Pub. Date: Sep. 21, 2017

(54) Alert assistance for survey mode ultrasound imaging

(71) Applicant: Siemens Medical Solutions USA, Inc., Malvern, PA (US)

(72) Inventors: Lei Sui, Medina, WA (US); Andrzej Milkowski, Issaquah, WA (US)

(21) Appl. No.: 15/075,013

(22) Filed: Mar. 18, 2016

(51) Int. Cl. A61B 8/08; A61B 8/00

(52) U.S. Cl. CPC A61B 8/5223; A61B 8/485; A61B 8/5207; A61B 8/4483; A61B 8/4461; A61B 8/486; A61B 8/488; A61B 8/481

(57) ABSTRACT

For alert assistance for an ultrasound scanner, computer-assisted detection is applied as the patient is scanned. The user may be notified of any detected objects so that the user gathers more information when appropriate. An automated system may be configured to return to scan any detected objects. Information is gathered as part of the work flow for that given examination of the patient based on the detection. A mechanical property of the object is derived from the extra information, resulting in further information that may be used to avoid a return visit and/or increase sensitivity in survey mode scans.

### BACKGROUND

The present embodiments relate to ultrasound imaging. In ultrasound imaging, users ‘survey’ organs for suspicious areas. A survey is typically done by moving the transducer to scan different planes through an organ. The goal is to identify any suspicious objects by viewing the sequence of images created while surveying. Occasionally, the user moves the transducer or reviews the images more quickly, increasing the likelihood of missing an object. Image persistence and/or related motion blur may contribute to the user missing a suspicious area. Users may reduce survey speed or repeatedly scan one region if the user is suspicious to increase sensitivity, but this requires time that may not be available. Either a suspicious area is not identified (lowering sensitivity) or scanning time is increased to confirm imaging results.

For automated surveys, such as by a volume scanner, the system sweeps the patient once to generate a sequence of images. The review for suspicious objects may occur after a patient examination is complete. As a result, the patient may have to return for another examination for more detailed scan of any suspicious regions. This “call back” approach may be inefficient and costly.

### BRIEF SUMMARY

By way of introduction, the preferred embodiments described below include methods, instructions, and systems for alert assistance for an ultrasound scanner. Computer-assisted detection is applied as the patient is scanned. The user may be notified of any detected objects so that the user gathers more information when appropriate. An automated system may be configured to return to scan any detected objects. Information is gathered as part of the work flow for that given examination of the patient based on the detection. A mechanical property of the object is derived from the extra information, resulting in further information that may be used to avoid a return visit and/or increase sensitivity in survey mode scans.

In a first aspect, a method is provided for alert assistance for an ultrasound scanner. A patient is scanned with an ultrasound transducer of the ultrasound scanner in a survey mode in which the ultrasound transducer is moving relative to the patient. Computer-assisted detection is applied by the ultrasound scanner to each of a sequence of frames acquired by the scanning. The computer-assisted detection of the ultrasound scanner identifies an object in a first of the frames. In response to the identification of the object in the first frame, a mechanical property of the object is measured. An image is generated with an alert identifying the first frame and the measured mechanical property.

In a second aspect, a non-transitory computer readable storage medium has stored therein data representing instructions executable by a programmed processor for alert assistance in ultrasound imaging. The storage medium includes instructions for: generating ultrasound images of a patient with an ultrasound transducer while the ultrasound transducer is moving along the patient; applying detection of a target to the ultrasound images during acquisition of the ultrasound images; recording a location of the ultrasound transducer in response to detection of the target in one of the images; notifying of the detection; acquiring data representing the object in response to the detection and using the location; deriving a value of a characteristic of the target from the data; and presenting the value.

In a third aspect, a system is provided for alert assistance in ultrasound imaging. A robot connects with a transducer and is configured to move the transducer in a pre-determined scan pattern. A transmit beamformer and a receive beamformer are configured to scan, with the transducer, a patient with ultrasound while the robot moves the transducer. A processor is configured to apply computer-assisted detection to results from the scan, to cause the robot to return the transducer to a location of detection by the computer-assisted detection after completing the pre-determined scan pattern, and to derive a mechanical property of tissue based on information acquired with the transducer returned to the location. A display is operable to display the mechanical property of the tissue with a flag for the results for the location.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for alert assistance for an ultrasound scanner;

FIG. 2 illustrates an example of a survey mode with an object identified in one image for measuring a mechanical property; and

FIG. 3 is one embodiment of a system for alert assistance in ultrasound imaging.

### DETAILED DESCRIPTION OF THE DRAWINGS AND PRESENTLY PREFERRED EMBODIMENTS

The survey mode workflow is improved in medical ultrasound imaging. During or survey mode, the sonographer or a robot moves the transducer to survey an organ and/or to locate a region of interest. In survey, the motion of the transducer allows scanning different regions of the patient to survey that organ or range of regions of the patient for any suspicious objects. For example, a transducer is moved along a patient's breast in order to survey the breast for any tumors or cysts. The patient may be surveyed to find a region of interest. The transducer is moved to find an organ or location of interest for more intensive imaging. Part of the patient is surveyed in order to find the location of interest.

An alert is provided in survey mode to assist in user finding of objects in the patient. The alert is a notification to the user of detection of the object, a flag or recording of the data or image representing the object, an indication of the location of the transducer for scanning the object, a highlight on an image during review, or other real-time or post procedure alert. The user is assisted by the alert, such as the alert helping to avoid missing the object and/or indicating where to scan or measure.

In one robotic scanning embodiment, image data is supplemented with acoustic and/or mechanical properties in real-time. A robot scans in a predefined standard. An algorithm runs in the background to recognize features, such as dense areas in a breast examination. For any recognized features, the imaging posture (e.g., transducer position and angle) is recorded and a user is notified. Alerts, such as labels for images with suspicious objects and/or saving of the suspected images in addition to the conventional CINE loop, are provided to the user. Acoustic and/or mechanical properties of the target are then examined by returning to the target area, automatically or manually, after scanning with the predefined standard. Alternatively, the property is derived from the saved data from the survey scan. The investigation of acoustic and/or mechanical properties may be done with a different device (i.e., other than the ultrasound scanner) operated by the robot or a person or done with the same ultrasound system. Suspected objects labeled with associated mechanical properties are highlighted in one or more images and/or volume renderings.

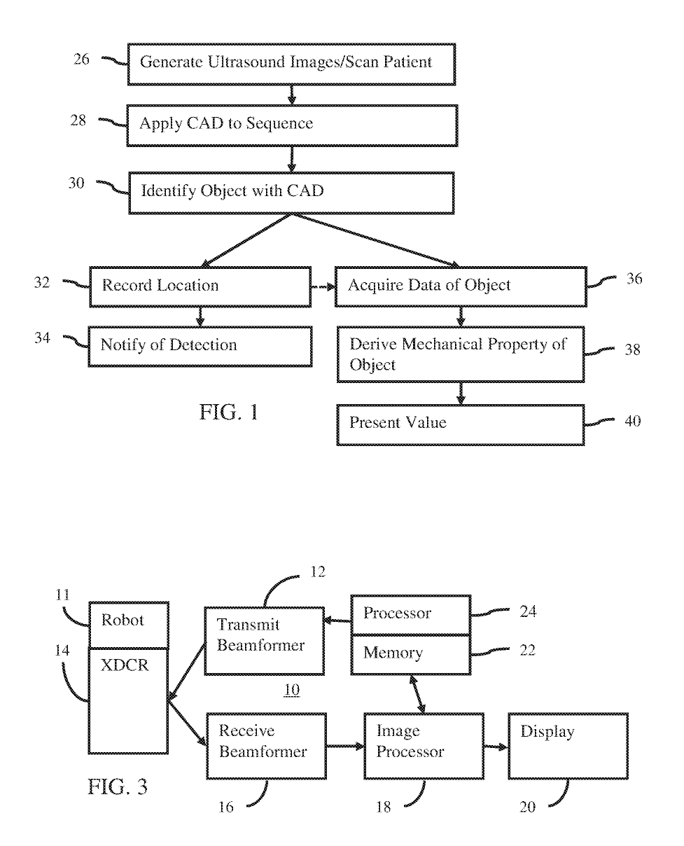

FIG. 1 shows a method for alert assistance for an ultrasound scanner. During a survey mode of operation where the transducer moves to survey the patient, computer-assisted detection indicates locations at which a mechanical property should be measured. This alerts the user to a suspicious region and makes gathering needed data part of the workflow while both limiting the likelihood of a patient leaving and having to come back for another examination and increasing sensitivity. The method improves medical imaging and scanning to gather information usable by a physician for medical diagnosis.

The method is implemented by the system of FIG. 3 or a different system. For example, the ultrasound imaging system generates ultrasound images using beamformers and a transducer. A processor, controller, or image processor of an ultrasound imaging system applies computer-assisted detection to identify an object, record the location of the transducer and/or object, and notify of the detection. The ultrasound imaging system or other device acquires data for the object, derives a mechanical property of the object, and presents the value as an alert to assist the user. Beamformers, memory, detectors, and/or other devices may be used to acquire the data, perform one or more of the acts, and/or output the data. The processor may control the devices to perform the method of FIG. 1.

Additional, different, or fewer acts may be provided. For example, the method is performed without recording the location in act 32 or notification in act 34. As another example, the method is performed without presenting the value in act 40. Acts 34 and 40 may be a same act, such as the presentation of the value of act 40 being the notification of the detection of act 34.

The acts are performed in the order described or shown (e.g., top to bottom), but may be performed in other orders. Acts 32 and/or 34 may occur before, after, or simultaneously with any of acts 36, 38, and/or 40.

In act 26, the ultrasound scanner generates ultrasound images of the patient. The images are generated by scanning the patient. Alternatively, the images are generated by loading frames of data from a memory. The images were generated from a previous scan of the patient stored in memory.

Any type of ultrasound images may be generated, such as B-mode, flow mode (e.g., Doppler velocity or power), contrast agent, harmonic, pulsed wave Doppler (i.e., spectral Doppler), M-mode, Doppler tissue (i.e., tissue motion), or other ultrasound imaging mode representing acoustic interaction with the patient. The different modes detect different types of information, such as intensity of acoustic return (e.g., B-mode and M-mode) or velocity (e.g., flow mode or Doppler tissue).

To acquire the images, the ultrasound transducer is moved along the patient. The movement is along a skin of the patient, but may be along a vessel or organ within the patient (e.g., scanning with a probe or catheter). The overall process for scanning and imaging includes placing the transducer on the patient, rotating and/or translating the transducer to survey the patient, scanning while the transducer moves, and generating images as part of the survey. In this process, the transducer may or may not be lifted from the patient. The images are generated while the transducer is moved along the patient and/or while scanning with the transducer. Alternatively, frames of data are acquired while surveying or moving the transducer and images are generated at a later time.

During the survey mode, the scanning occurs as the transducer is moved relative to the patient. The movement is of the transducer, rather than internal parts of the patient moving. The transducer moves relative to tissue of the patient contacting the transducer. The movement of the transducer causes the ultrasound scanner to scan different planes or volumes of the patient at different times. In this way, the patient is surveyed over time. The sequence of scans acquires data representing different parts of the patient.

In one embodiment, the transducer is moved manually by the user or sonographer. The user holds the transducer and slides and/or rotates the transducer to survey the patient, such as translating the transducer to scan different planes through an organ (e.g., breast).

In another embodiment, the transducer is moved automatically. A robot scans the patient. The robot controls the position and/or movement of the transducer. For example, a robotic arm moves one or more joints to translate and/or rotate the transducer. In another example, the robot includes a chain drive, screw drive, gearing, rack and pinion, or other transmission to move the transducer along a flat or curved plate or surface. Any now known or later developed robotic system with a motor may be used to move the transducer without user force being applied to move during the scanning. Automated breast volume or other scanning may use a robot.

The robot and/or user move the transducer over a predefined region. A pre-determined pattern is used for scanning. For example, the robot moves the transducer to different points, over a range of rotation, and/or over a range of translation to scan. The pre-determined pattern defines the spacing, speed, ranges, and/or steps used in the movement of the transducer. The time and/or position at which each scan occurs may be set. Alternatively, the scanning is continuous or periodic regardless of position while the transducer is moved. In other embodiments, the survey occurs over any region without pre-determination, such as during a manual survey.

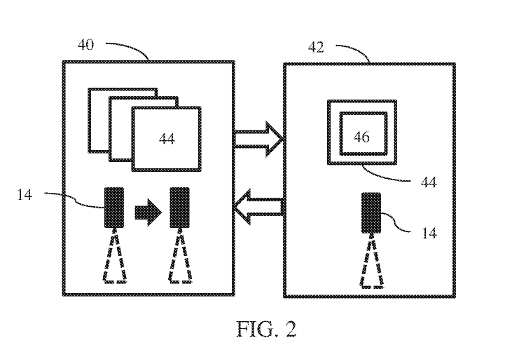

FIG. 2 shows an example of survey mode acquisition 40. The transducer 14 is moved, resulting in frames 44 of data being acquired. The different frames 44 represent different, parallel planes in this example.

During the survey mode of imaging, a large amount of data is collected. Each scan of a plane or volume provides a frame of data. Frames of data are provided for the various positions of the transducer. An image may be generated from each frame. With persistence or other compounding, images may be generated from multiple frames. Tens, hundreds, or thousands of frames and/or images are created for surveying the patient in one examination (e.g., one implementation of the predetermined pattern and/or during a given visit of a patient to the sonographer). The large number of frames or images poses a challenging problem to review the data and identify suspicious targets.

In act 28, the ultrasound scanner or an image processor applies computer-assisted detection to each of the frames or images of the sequence acquired by the scanning. The images may be display values (RGB) or scalar data used to generate display values. The frames are images or data at other stages of processing. The scanning provides a sequence of frames and/or images. The computer-assisted detection and other processing described herein is applied to the frames and/or images.

Any now known or later developed computer-assisted detection may be applied. For example, pattern matching is used to determine whether a pattern indicative of a tumor or cyst is located in the frame or image. As another example, thresholding, segmentation, or other image processing is applied. For more rapid detection, a machine-learnt detector may be applied. The machine-learnt detection is a Bayesian network, support vector machine, neural network, or other detector relating input features from the frame or image (e.g., steerable features or Haar wavelets) to suspicious objects.

Where the computer-assisted detection is based on machine learning, a self-learning or feedback learning may be used. As a physician reviews detected objects and indicates whether the object is suspicious or of interest, this information may be used as further training data to re-learn or update the detector with additional ground truth.

The computer-assisted detection is applied to all of the frames. In other embodiments, the detection is applied to fewer than all the frames. Where the ultrasound scanner may be configured for a survey mode, the detection may be applied to all frames. In other approaches, the detection is applied to frames associated with transducer movement. Using a sensor on the transducer, knowledge of operation of the robot, and/or data correlation to detect movement of the transducer, the ultrasound scanner may apply the detection to frames associated with movement and not to frames where the transducer is stationary.

The computer-assisted detection is applied during the acquisition of the frames or images. The application is in real-time. The period to process a frame is equal to or less than the period to acquire a new frame, allowing the application to occur in real-time, such as completing application of the detection within a second of creating the scan. In other embodiments, the application occurs in a post process after scanning is complete. Combinations of application of detection during the scanning and as a post process may be used.

Different computer-assisted detectors may detect different types of objects and/or objects in different situations. Multiple detectors may be applied to each frame. In one embodiment, the detectors to be applied are selected based on the type of examination. For example, the user configures the ultrasound scanner for a breast examination. A detector or detectors for detecting suspicious objects (e.g., tumors and/or cysts) in the breast are selected and applied. As another example, the user configures the ultrasound scanner for breast examination to detect cancer. A detector or detectors for detecting cancer objects in the breast are selected. The selection is automatic by the processor, or the user selects the detectors.

In act 30, one or more objects are identified by the image processor or ultrasound scanner. One or more objects are identified in any number of the frames or images. Some frames may be determined to not include any of the target objects. For each or some of the acquired images, targets are recognized. For example, the computer-assisted detection locates suspicious objects in five of a thousand images. A tumor or other object is identified. No objects may be detected.

FIG. 2 shows an example. In the detection operation 42, the object 46 is detected in one of the frames 44. The transducer 14 is positioned at a given location relative to the patient for acquiring that frame 44 with the object 46.

Where the computer-aided characterization runs real time, the suspicious areas are highlighted during real-time scanning while surveying the patient. The objects are identified during the scanning. For post processing, the suspicious areas are located after the scanning is complete.

Where persistence, steered spatial compounding, or other compounding is used, the identification may be in images or frames not displayed as part of the persistence or compound imaging. For example, the detector is applied to component frames used to persist or spatially compound. For the survey imaging, the persisted (e.g., temporally filtered) or spatially compounded (e.g., combination of frames with different steering directions) images are displayed without displaying the component frames or images. The detection is applied to the component frames or images and/or the frames or images as compounded. Where the detection is positive, the component frame or image may be displayed without the compounding or persistence. Alternatively or additionally, detection or not in multiple of the component frames may be used to indicate confidence in the detection.

In act 32, the ultrasound scanner or image processor records a location of the ultrasound transducer in response to detection of the target in one of the images. The detector detects an object in a frame. That frame corresponds to a given position of the transducer. The position of the transducer for the detected frame is recorded. The recording may be performed for all the frames so that the location may be looked-up for any frames with a detected object in response to the detection. Alternatively, the recording occurs only for frames where the object is detected.

The recording is electronic or physical. The transducer location may be tracked, such as by an optical system, a magnetic system, or other transducer tracking. The lateral position and orientation of the transducer is tracked. These systems electronically record the location based on the calibrated frame of reference. Inertial tracking may be used. For physical recording, the position of the transducer is marked physically. For example, the transducer excretes a colored ink or dye upon detection so that the location is marked on the patient and/or in the acoustic coupling gel. As another example, a marking is indicated on a frame or guide for the transducer.

The frame or image may likewise be recorded with or without the identified object. A set of frames or images with detected objects is created for reference by a user. Alternatively, the identity of the frame or image in the sequence of images or frames is recorded. The frame or image may be recalled from the sequence.

In act 34, the image processor or ultrasound scanner notifies of the detection. The notification is an output to the user. Any output may be used. In one embodiment, the output is visual on a display. Text or a symbol indicating that an object was detected is output. The image with the detected object may be output with or without highlighting the detected object in the image as notification. Another example output is dying or staining the patient or acoustic gel.

In one embodiment, the notification includes the location. The location is provided as coordinates, a spatial position on the patient (e.g., shown on a graphic of or marked on the patient), feedback indicating whether the transducer is moving towards or at the location, or other indicator of location.

In additional or alternative embodiments, the notification is provided by audio. A noise is made upon detection. The noise indicates that the transducer is at the location in real-time implementation. Alternatively, the noise is provided as feedback to guide the user to position the transducer at the location (e.g., periodic tone with greater frequency for closer to the location).

Other notifications may be used, such as a combination of audio and video. Tactile (e.g., vibration of the transducer) and/or smell may be used. The notification is of the occurrence, the location of the transducer and/or scan plane, the location of the object in the image, other information about the detection, and/or combinations thereof.

The notification is provided upon occurrence of the detection. Once detected, the notification is output so that the transducer may be maintained at the position to gather additional information in act 36. For a robotic implementation, the notification may be used so that the patient can be informed that the examination may take longer than expected. In alternative or additional embodiments, the notification is provided seconds, minutes, hours, or any period after occurrence of the detection. For example, after the scan in the pre-determined format is complete, the robotic system notifies that further scanning is to occur due to detection of one or more objects. As another example, a physician loads the results of the examination for later review. The physician is notified that detection occurred. This notification may be a set of flagged images with detected objects, the flags, or other information.

In one embodiment, the ultrasound scanner or image processor provides information about the detection in addition or other than the location of the transducer, the occurrence, and/or the location of the object in the image. For example, a confidence in the detection is provided. Machine-learnt classifiers may provide confidence information. Other sources of confidence may be used, such as a degree of correlation of the image with a template or fuzzy logic-based confidence. Where multiple images or frames representing a same or overlapping fields of view are provided (e.g., slow moving transducer and/or compounding type of imaging), the confidences from the multiple frames or images may be combined (e.g., averaged) to provide a confidence for the particular object.

This confidence is output to the user as a percentage, color-coding, or other indicator. In one embodiment, different colors and/or intensities of the highlighting of the object in the image represent different ranges or levels of confidence. Other indicators of confidence may be provided, such as the order of presenting the images. The stored images are provided with the most confident detections first, last, or in an order ranked by confidence.

In alternative embodiments, the user is not notified. Instead, acts 36, 38, and 40 are performed without a separate notification. Act 40 may be a form of notification in the sense that providing a value of a mechanical property indicates that an object was detected.

In act 36, the ultrasound scanner, another scanner (e.g., x-ray), and/or laboratory testing equipment (e.g., robotic biopsy) acquires data representing the object in response to the detection. The same or different measuring tools (e.g., magnetic resonance, ultrasound, manual tap, or other tool) are used to acquire the data for a mechanical property. The data is acquired by measurement performed on the object in the patient. For example, the ultrasound scanner re-scans the object to gather different or additional data. Alternatively, the data is acquired from memory, such as using the frame or image data at the location (i.e., data used to detect the object) to derive further information.

The acquired data is for a mechanical property. Rather than just scanning for imaging (e.g., intensity of acoustic return (B-mode) and/or velocity or power of flow (flow mode)), data representing a mechanical property or characteristic of the object itself is acquired. For example, the elasticity, shear velocity, Young's modulus, strain, strain rate, or other parameterization of the object is measured. The measurement uses more than just a frame of data or image to derive the characteristics of the object. The measurement may be focused on the object and/or have a field of view smaller than for the generation of the images for the survey.

The image processor, ultrasound scanner, or other controller causes the acquisition of the data for the detected object. The acquisition is automated, semi-automatic, or manual. The controller may display instructions to the user so that the user acquires the data during the examination or given visit of the patient. The user moves the device for measurement. The controller may perform some operations automatically, such as measuring and/or positioning for measuring once the user activates and/or positions. The controller may locate for measuring (e.g., robotically move the transducer) and perform the measurement (e.g., scan for a mechanical property of the object) without user input of location, scanning, and/or activation. The data is acquired in real-time (e.g., during a same examination) or is performed later (e.g., manually off-line).

To acquire the data for the mechanical property, the location of the object is used. The ultrasound scanner, controller, or image processor uses the location at which the transducer was at when having scanned the object. That location of the transducer and the location of the object relative to the transducer based on the scan format are used to acquire the data. The data is acquired from the object, and the location of the transducer is used to indicate the where the object can be found. Using a marking and/or position sensing, the location information is used to position a device for measurement. As represented by the dashed arrow between acts 32 and 36, the recorded location of act 32 may or may not be used to guide the acquisition of further information about the object in act 36. The record of the location of the suspected images may be from the robotic posture, electromagnetic sensor, inertial locator, probe dropped marker, acoustic images themselves, and/or video recorded scanning.

For example, the robot positions the transducer to acquire data used for measuring the characteristic. A motor connected with the ultrasound transducer causes the transducer to return to a position for scanning the object. The position is a same position used during the survey from which the object was detected. The transducer is returned to the location at which the transducer was for scanning the patient to acquire the frame in which the object is detected. After completing the pre-determined scan pattern for the survey or by interrupting the pattern, the transducer is kept at or repositioned to scan the object again. The mechanical property is then measured.

In the manual survey embodiment, the user stops the survey, slows down the survey, and/or returns after surveying to acquire the data. For example, the transducer is stopped or returned to the location to acquire the data (e.g., perform shear wave measurements). As another example, the user temporally suspends the survey upon notification, holding the transducer in place. The transducer is then used to acquire further data.

In the automated or robotic survey embodiment, the robot stops the survey or returns the transducer to the location after completing the survey. Additional interrogation, such as elasticity imaging, is performed by the ultrasound scanner once the transducer is returned to the location corresponding to the detected object. The acquisition of the data, based on the real-time detection of the object, occurs as part of the examination and/or without the patient leaving the examination. The examination, including gathering information about the suspicious object or objects, occurs automatically, providing more or complete information at the end of the given appointment or examination of the patient. Both the survey and further information for diagnosis are acquired without the patient having to make multiple appointments or be examined multiple times. The detection and acquisition of data may provide for more compact reporting as well, such as sending the frames or images with detected objects and the acquired data with or without the survey so that the radiologist may focus on the information of interest.

In act 38, the image processor, ultrasound scanner, and/or other device acquiring the data in act 36 derives a value of a characteristic of the target from the acquired data. The acquired data is used to derive the mechanical property. The character of the object is measured as a mechanical property, but other characteristics of the object may be calculated.

In response to the identification or detection of the object, the mechanical property is measured. Any mechanical property or other characteristic may be derived. For example, the ultrasound scanner measures strain, strain rate, shear velocity, elasticity, or Young's modulus. These mechanical properties represent more than a response to energy from imaging. For strain and strain rate, multiple scans are provided to derive the strain due to motion of the tissue. For shear velocity, elasticity, or Young's modulus, tissue is displaced by acoustic or other force and the tissue response to the displacement or generated wave is measured. The characteristic of the object is derived from more than just imaging. Multiple scans may be used to then calculate displacement over time, which is used to derive a shear velocity or elasticity. The Young's modulus is derived from the shear velocity and/or elasticity. While the frame from the survey may be used, such as a reference for calculating displacement, other frames of data are acquired in act 36 for deriving the characteristic of the object.

In act 40, the ultrasound scanner, image processor, or other device presents the derived value or values. The value may be diagnostically useful and provides information in addition to imaging. The value is output to the user, such as outputting on a display. In one embodiment, a display of the image with the detected object highlighted is generated. The value of the characteristic is provided as an annotation, label, and/or in the image as text, a graph, bar, color coding, and/or brightness coding. By viewing the display, the image of the patient and object is provided as well as an indication of the mechanical property or properties of the object.

In one embodiment, the value is provided as or with the notification of act 34. In other embodiments, the value is provided separately from the notification, such as providing the value after notification and during a subsequent review for diagnosis and/or to confirm acquisition of the additional data in act 36. In either case, the user is alerted to the characteristic of the object, assisting in the survey.

Other outputs than to the display may be used. The value may be stored in the patient record, with the survey, and/or with recordings of the location, object, and/or frames with detected objects. The value is then displayed to the user during review.

Act 28 is applied to all or multiple frames or images of the sequence generated by scanning in the survey mode. Where objects are detected in multiple frames in act 30, the location and/or frames are stored in act 32 and separate notices are provided in act 34. In other embodiments, one notice with a list of locations, frames, images, or other information about detected objects is provided upon completion of the survey or later. Since act 32 is repeated for each object, a group of frames with detected objects is identified or gathered. This group may be provided separately from the survey for review or further analysis. Acts 36, 38, and 40 are performed for each object.

As the user reviews images of the detected objects with the derived values, the user may indicate that the detection of the object is accurate or not. For example, the value and/or image may show the user whether the object is or is not a tumor, cyst, or other object of interest. The user inputs whether the detection is accurate or not. This feedback is used as ground truth information. The image and the feedback are provided for machine learning to update the detector applied in act 28.

FIG. 3 shows one embodiment of a system 10 for alert assistance in ultrasound imaging. The system is alert in applying object detection, providing assistance. Alternatively or additionally, the system alerts the user to object detection. The ultrasound system is configured for surveying a patient. During the survey, the acquired data is analyzed by the system for objects of interest. If any objects are detected, then the system acquires mechanical property information about the object. Rather than just providing images from a survey, the images and mechanical property information for objects of interest are provided.

The system 10 is a medical diagnostic ultrasound imaging system. In alternative embodiments, the system 10 is a personal computer, workstation, PACS station, or other arrangement at a same location or distributed over a network for real-time or post acquisition imaging through connection with beamformers 12, 16 and transducer 14.

The system 10 implements the method of FIG. 1, the approach of FIG. 2, or other methods. The system 10 includes a robot 11, a transmit beamformer 12, a transducer 14, a receive beamformer 16, an image processor 18, a display 20, a memory 22, and a processor 24. Additional, different or fewer components may be provided. For example, a user input is provided for manual or assisted designation of a region of interest within a field of view for mixed mode imaging and/or for configuring the ultrasound system 10 for mixed mode imaging. As another example, the robot 11 is not provided.

The robot 11 is a motor and a device for moving the transducer 14 with force from the motor. The robot 11 may have any number of arms and joints. In other embodiments, the robot 11 is a tray supporting a transducer 14 along rails where the motor moves the transducer 14 along the rails. Gears, chains, screw drive, or other devices may be provided for translating the motor force (e.g., rotation) to movement of the transducer 14.

Under control of the processor 24 or other controller, the robot 11 is configured to move the transducer in a pre-determined pattern. The movement is constant or by steps. Any pattern may be used, such as moving the transducer 14 along a line from a starting point to a stopping point. Another pattern moves the transducer 14 from point to point in a regular grid on the patient. The pattern may or may not include tilting or rotating the transducer 14. The robot 11 may be configured to move the transducer 14 to particular locations based on detection of objects. This further movement occurs after completion of movement for the pre-determined pattern.

The robot 11 connects with the transducer 14. The connection is fixed or releasable. For example, a gripper of the robot 11 holds the transducer 14, but may release the transducer 14. As another example, the transducer 14 is fixed by screws, bolts, latches, or snap fit to a holder of the robot 11.

The transmit beamformer 12 is an ultrasound transmitter, memory, pulser, analog circuit, digital circuit, or combinations thereof. The transmit beamformer 12 is configured to generate waveforms for a plurality of channels with different or relative amplitudes, delays, and/or phasing. The waveforms are generated and applied to a transducer array with any timing or pulse repetition frequency. For example, the transmit beamformer 12 generates a sequence of pulses for B-mode scanning in a linear, sector, or Vector® format. As another example, the transmit beamformer 12 generates a sequence of pulses for color flow scanning, such as pulses for forming 2-12 beams in an ongoing flow sample count per scan line for a region of interest within a B-mode field of view. In yet another example, the transmit beamformer 12 generates pulses for elasticity or shear imaging. The transmit beamformer 12 may generate a beam for acoustic radiation force impulse. The intensity of the beam causes a shear wave or longitudinal wave to be generated from the focal point. The transmit beamformer 12 then generates beams for tracking the tissue response to the generated wave.

The transmit beamformer 12 connects with the transducer 14, such as through a transmit/receive switch. Upon transmission of acoustic waves from the transducer 14 in response to the generated waves, one or more beams are formed during a given transmit event. The beams are for B-mode, color flow mode, elasticity, shear wave, and/or other modes of imaging. A sequence of transmit beams are generated to scan a one, two or three-dimensional region. Sector, Vector®, linear, or other scan formats may be used. For each position of the transducer 14 or as the transducer 14 moves, a complete scan of the region is performed. Multiple such complete scans are performed with the transducer 14 at different locations or ranges of locations.

The transducer 14 is a 1-, 1.25-, 1.5-, 1.75- or 2-dimensional array of piezoelectric or capacitive membrane elements. The transducer 14 includes a plurality of elements for transducing between acoustic and electrical energies. For example, the transducer 14 is a one-dimensional PZT array with about 64-256 elements.

The transducer 14 connects with the transmit beamformer 12 for converting electrical waveforms into acoustic waveforms, and connects with the receive beamformer 16 for converting acoustic echoes into electrical signals. The transducer 14 transmits beams. To form the beams, the waveforms are focused at a tissue region or location of interest in the patient. The acoustic waveforms are generated in response to applying the electrical waveforms to the transducer elements. For scanning with ultrasound, the transducer 14 transmits acoustic energy and receives echoes. The receive signals are generated in response to ultrasound energy (echoes) impinging on the elements of the transducer 14.

The receive beamformer 16 includes a plurality of channels with amplifiers, delays, and/or phase rotators, and one or more summers. Each channel connects with one or more transducer elements. The receive beamformer 16 applies relative delays, phases, and/or apodization to form one or more receive beams in response to each transmission for imaging. Dynamic focusing on receive may be provided. Relative delays and/or phasing and summation of signals from different elements provide beamformation. The receive beamformer 16 outputs data representing spatial locations using the received acoustic signals. In alternative embodiments, the receive beamformer 16 is a processor for generating samples using Fourier or other transforms.

The receive beamformer 16 may include a filter, such as a filter for isolating information at a second harmonic, transmit (i.e., fundamental), or other frequency band relative to the transmit frequency band. Such information may more likely include desired tissue, contrast agent, and/or flow information. In another embodiment, the receive beamformer 16 includes a memory or buffer and a filter or adder. Two or more receive beams are combined to isolate information at a desired frequency band, such as a second harmonic, cubic fundamental, or other band.

The receive beamformer 16 outputs beam summed data representing spatial locations. Data for a single location, locations along a line, locations for an area, or locations for a volume are output. The data beamformed in response to a complete scan of a region is a frame of data. As the transducer moves, such as by the robot 11, the complete scan of each region is performed, providing frames of data representing spatially different fields of view.

The image processor 18 is a B-mode detector, Doppler detector, pulsed wave Doppler detector, correlation processor, Fourier transform processor, filter, other now known or later developed processor for implementing an imaging mode, or combinations thereof. The image processor 18 provides detection for the imaging modes, such as including a Doppler detector (e.g., estimator) and a B-mode detector. A spatial filter, temporal filter, and/or scan converter may be included in or implemented by the image processor 18. The image processor 18 outputs display values, such as detecting, mapping the detected values to display values, and formatting the display values or detected values into a display format. The image processor 18 receives beamformed information and outputs image data for display.

The processor 24 is a control processor, general processor, digital signal processor, graphics processing unit, application specific integrated circuit, field programmable gate array, network, server, group of processors, data path, combinations thereof, or other now known or later developed device for detecting objects in images and controlling the ultrasound system 10 to image accordingly. The processor 24 is separate from or part of the image processor 18. As a separate device, the processor 24 requests, receives, accesses, or loads data at any stage of processing (e.g., beamformed, detected, scan converted, display mapped or other stage) for detecting and controlling. The processor 24 is configured by software and/or hardware to perform or cause performance of the acts of FIG. 1.

The processor 24 is configured to apply computer-assisted detection to results from the scan. The frames of data from the receive beamformer 16 and/or any stage of processing of the image processor 18 are input to the computer-assisted detection. For example, Haar wavelets, gradients, steerable, and/or other features are calculated from each frame of data. These features are input as a feature vector into a machine-learnt detector. Based on these features, the detector indicates whether or not the object is in the image, a location of any object of interest in the image, and/or a confidence in any detection. In another example, a template or pattern representing the object of interest is correlated by the processor 24 with the frame of data in various relative positions. If a sufficient correlation is found, an object of interest is detected. Any now know or later developed computer-assisted detection may be used.

The processor 24 is configured to control the robot 11. The robot 11 keeps or returns the transducer 14 to a same or similar (e.g., with an overlapping field of view) position as when an object was scanned. Based on detection of an object of interest, the processor 24 determines the location of the transducer 14 at the time of scanning for the frame with the object. The transducer 14 is halted at that position to acquire data for measuring a mechanical property. Alternatively, the pre-determined scan pattern or movement pattern of the transducer 14 by the robot 11 is completed, and then the processor 24 causes the robot 11 to return the transducer 14 to the location.

In other embodiments, the processor 24 generates a notification to the user. For example, a notification is presented on the display 20. As another example, the transducer 14 is controlled to mark (e.g., dye) the location on the patient. The processor 24 may be configured to provide feedback to the user to manually position the transducer 14, such as indicating an amount and direction of movement, proximity to the location, or other communication leading to the user being able to position the transducer 14 at a same location or hold the transducer 14 at a current location for acquiring additional data.

The processor 24 is configured to record the location, the frame with the object, the detection of the object, confidence of detection, and/or other information. The information is recorded with or separate from the image results of the survey.

The processor 24 is configured to derive a mechanical property of tissue. The beamformers 12, 16 are controlled to acquire additional data about the object once the transducer 14 is in the correct location. For example, elasticity or shear wave tracking is performed. The processor 24 uses the acquired data to calculate a mechanical property of the detected object.

The processor 24 or image processor 18 generates and outputs images or values to the display 20. For example, B-mode or mixed mode (e.g., B-mode and flow mode) images are output. Text, numerical indication, or graphic may be added and displayed to the user. A graph may be displayed. For example, an annotation marking a detected object, a flag indicating the image as including a detected object, the derived value of the mechanical property of the object, confidence of detection, or other object related information is output. The images associated with detected objects are flagged, such as providing the images on the display 20 separate from CINE presentation of the survey. The output of the value and/or object highlighting may likewise flag an image as including a detected object. Location information, such as of the transducer 14, may be output.

During the survey, the display 20 displays images representing different fields of view or regions in the patient. Flags, alerts, notification, values, or other information may be displayed at that time or during a later review.

The display 20 is a CRT, LCD, monitor, plasma, projector, printer, or other device for displaying an image or sequence of images. Any now known or later developed display 20 may be used. The display 20 is operable to display one image or a sequence of images. The display 20 displays two-dimensional images or three-dimensional representations.

The image processor 18, processor 24, the receive beamformer 16, and the transmit beamformer 12 operate pursuant to instructions stored in the memory 22 or another memory. The instructions configure the system for performance of the acts of FIG. 1. The instructions configure the image processor 18, the processor 24, the receive beamformer 16, and/or the transmit beamformer 12 for operation by being loaded into a controller, by causing loading of a table of values (e.g., elasticity imaging sequence), and/or by being executed.

The memory 22 is a non-transitory computer readable storage media. The instructions for implementing the processes, methods and/or techniques discussed herein are provided on the computer-readable storage media or memories, such as a cache, buffer, RAM, removable media, hard drive or other computer readable storage media. Computer readable storage media include various types of volatile and nonvolatile storage media. The functions, acts, or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing, and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I(We) claim:

1. A method for alert assistance for an ultrasound scanner, the method comprising:
scanning a patient with an ultrasound transducer of the ultrasound scanner in a survey mode in which the ultrasound transducer is moving relative to the patient;
applying computer-assisted detection by the ultrasound scanner to each of a sequence of frames acquired by the scanning;
identifying an object in a first of the frames with the computer-assisted detection of the ultrasound scanner;
measuring, in response to the identification of the object in the first frame, a mechanical property of the object; and
generating an image with an alert identifying the first frame and the measured mechanical property.

2. The method of claim 1 wherein scanning comprises scanning with the movement of the ultrasound transducer controlled by a motor.

3. The method of claim 1 wherein scanning comprise scanning with the movement of the ultrasound transducer performed manually by a user.

4. The method of claim 1 wherein scanning comprises robotic scanning and wherein measuring comprises robotic measuring.

5. The method of claim 1 wherein applying comprises applying the computer-assisted detection as a machine learnt detector of the object operable to process the frames of the sequence while the scanning is performed.

6. The method of claim 1 wherein identifying comprises identifying a tumor in the first frame and identifying no tumor in a plurality of other frames of the sequence.

7. The method of claim 1 wherein scanning comprise scanning in a pre-determined pattern for the sequence, and wherein measuring comprises controlling a motor connected with the ultrasound transducer to return the ultrasound transducer to a position for the first frame after the pre-determined pattern is complete and scanning for the mechanical property with the ultrasound scanner.

8. The method of claim 1 wherein measuring comprises deriving the mechanical property from, at least in part, the first frame.

9. The method of claim 1 wherein measuring comprises measuring strain, strain rate, shear velocity, elasticity, or Young's modulus.

10. The method of claim 1 wherein measuring comprises deriving a characteristic of the object other than response to energy from imaging.

11. The method of claim 1 wherein generating comprises generating the image with the alert being a flag of a location of the ultrasound transducer for scanning the object.

12. The method of claim 1 further comprising:
identifying objects in other frames of the sequence; and
storing the first frame and the other frames.

13. The method of claim 1 wherein generating comprises generating the image from the first frame with the alert comprising an indication that the object is detected in the image.

14. A non-transitory computer readable storage medium having stored therein data representing instructions executable by a programmed processor for alert assistance in ultrasound imaging, the storage medium comprising instructions for:
generating ultrasound images of a patient with an ultrasound transducer while the ultrasound transducer is moving along the patient;
applying detection of a target to the ultrasound images during acquisition of the ultrasound images;
recording a location of the ultrasound transducer in response to detection of the target in one of the images;
notifying of the detection;
acquiring data representing the object in response to the detection and using the location;
deriving a value of a characteristic of the target from the data; and
presenting the value.

15. The non-transitory computer readable storage medium of claim 14 wherein applying the detection comprises applying a computer-assisted detection specific to a type of examination for which the scanning is performed.

16. The non-transitory computer readable storage medium of claim 14 wherein presenting the value comprises generating a display of the one image with the target highlighted and including the value of the characteristic.

17. The non-transitory computer readable storage medium of claim 14 wherein acquiring the data comprises using the image, re-scanning the patient at the location, or both, and wherein deriving the value comprises deriving a mechanical property as the characteristic.

18. The non-transitory computer readable storage medium of claim 14 wherein recording the location comprises recording a position of the ultrasound transducer electronically or physically.

19. The non-transitory computer readable storage medium of claim 14 wherein notifying comprises indicating the location to a user.

20. A system for alert assistance in ultrasound imaging, the system comprising:
a transducer;
a robot connected with the transducer and configured to move the transducer in a pre-determined scan pattern;
a transmit beamformer and a receive beamformer configured to scan, with the transducer, a patient with ultrasound while the robot moves the transducer;
a processor configured to apply computer-assisted detection to results from the scan, to cause the robot to return the transducer to a location of detection by the computer-assisted detection after completing the pre-determine scan pattern, and to derive a mechanical property of tissue based on information acquired with the transducer returned to the location; and
a display operable to display the mechanical property of the tissue with a flag for the results for the location.
